(19) United States
(12) Patent Application Publication
(10) Pub. No.: US 2008/0289637 A1
(43) Pub. Date: Nov. 27, 2008

(54) DEVICE FOR TREATING SNORING SOUNDS, INTERRUPTIONS IN BREATHING AND OBSTRUCTIVE SLEEP APNEA

(75) Inventor: Arthur WYSS, Bern (CH)

Correspondence Address: PEARNE & GORDON LLP, 1801 EAST 9TH STREET, SUITE 1200, CLEVELAND, OH, 44114-3108, US

(73) Assignee: VELUMOUNT GMBH, Bern (CH)

(21) Appl. No.: 12/187,582

(22) Filed: Aug. 7, 2008

Related U.S. Application Data
(63) Continuation of application No. 12/033,340, filed on Feb. 19, 2008, now abandoned.
Application No. 12/033,340 is a continuation of application No. PCT/EP2006/065033, filed on Aug. 3, 2006.

(30) Foreign Application Priority Data
Aug. 19, 2005 (CH) 2005CH-01364

(51) Int. Cl. A61F 5/56

(52) U.S. Cl. 128/848

(57) ABSTRACT

The invention relates to a device (1) for treating snoring sounds, interruptions in breathing and obstructive sleep apnea (OSA) and for diagnosing and treating obstructions and vibrations in the pharyngeal region, with the device (1) being used intra-orally. The device (1) is comprised of at least one oral transverse (2a, 2b) and of means (3) which are placed at the rear end of the oral transverse (2a, 2b) for stabilizing the velum; the means (3) can be individually adapted to the patient's anatomy; during intra-oral use of the device (1), the velum is held at a distance from the pharyngeal wall and the connective tissue in the upper pharynx behind the velum is stabilized in order to prevent obstructions. Different means can advantageously complement the device and serve as holding device for sensors for examining and treatment purposes. The invention also relates to a method for adapting a device (1) according to one of the cited claims to the velum of a patient and to a method for recording measured values.

### RELATED APPLICATIONS

The present application is a continuation of U.S. patent application Ser. No. 12/033,340, filed on Feb. 19, 2008, which is a continuation of international application PCT/EP2006/065033, filed on Aug. 3, 2006, which claims priority of Swiss patent application 2005CH-01364, filed Aug. 19, 2005, all of which are incorporated herein by reference.

### FIELD OF THE INVENTION

The invention relates to a device for treating snoring sounds, interruptions in breathing and obstructive sleep apnea (OSA) according to the independent device claim, to a method for adapting a device to the velum of a patient according to the method claim 18 and to a method for recording measured values with a device corresponding to method claim 25.

### DESCRIPTION OF RELATED ART

Sleep disorders are, for various reasons, widely spread in today's society. Snoring is also one of these disorders. Habitual snoring represents a social problem as it can be a very disturbing nuisance for the partner or other people sleeping in the same room. Habitual snoring is not detrimental to the health of the afflicted person, but is already a first sign for a narrowing of the air influx during sleep. With increasing age, this narrowing can cause health deterioration. A sleep apnea can evolve.

Several devices for influencing snoring and obstructive sleep apnea are known in the prior art. These means however have practically not been able to impose themselves on the market for lack of broad effectiveness.

Mandibular protrusion splints can alleviate snoring and obstructive sleep apnea, but can influence negatively the orthodontia and orthognathia. Furthermore, their adjustment depends on the dentition.

Surgical interventions can bring relief but are however, like all operations, fraught with risk.

NCPAP devices (nasal continuous positive airway pressure), with which the airways are kept open during the night with continuous overpressure and breathing is made possible for the patients during sleep, are at present the choice therapy in the case of sleep apnea. These devices require sleeping with a mask, which again can cause social problems for the persons afflicted, since this in certain circumstances is not pleasant for the partner in the same room. Furthermore, these devices are voluminous and require maintenance and frequent replacement of used parts, which has a negative influence on the general health costs.

NCPAP devices are effective for many cases of sleep apnea, regardless of the location of the obstruction. Since many, especially younger patients, cannot come to terms with a NCPAP device for the above mentioned reasons, it would however be desirable to know the exact localization of the obstruction in order to be able to offer these persons less complex and also more cost-effective alternatives.

In order to assess sleep disorders, different methods are used nowadays.

Pulsoxymeric devices that are used for outpatients and inpatients and that measure the nightly oxygen saturation by means of an electrode affixed to a finger are established, but only show already serious breathing interruptions. Habitual snorers that only exhibit slight pathological breathing patterns cannot be detected like this. The cause of a breath flux disturbance cannot be determined.

Breathing monitors that can be used for outpatients measure the flux of air at the nasal inlets. With this method, it is not possible to determine the cause of a breathing flux disturbance. If the patient breathes through the mouth, the breath signal can additionally be too small to be able to be measured at the nasal inlet.

The inpatient sleep diagnosis is complex and cost-intensive. In this case, the degree of the breathing disorder is diagnosed exactly, however not the exact place of the triggering obstruction.

In order to determine the place of the obstruction, in order to be able to give the patient a specific therapy, the patient nowadays is put to sleep with a fast working anesthetic. By means of a glass fiber camera, vibrations and obstruction places in the pharynx can be elicited. This examination is complex and costly. Furthermore, it is seldom used nowadays.

Placing devices for therapeutic and diagnostic purposes directly in the throat without prior anesthesia of the patient has so far been hardly possible because of the natural gagging reflex. Placing tubes through the mouth in the pharynx to prevent snoring and sleep apnea as proposed in patent application WO-A1-98/09675 and DE-A1-195 01 363 or placing a hollow body with wall openings through the nose as described in DE-A1-102 40 725 therefore cannot be expected for a large part of the people afflicted.

In FR-A1-2,838,046, a flat appliance is described that is placed at the front around the incisors and fastened there, that lies flat in the middle between palate and tongue, and aligned in the middle behind the tongue, descending step-wise in the throat and resting against the throat's back wall. This concept can certainly prevent the collapse of the tongue backwards, but has no influence on the places of obstruction in the middle and upper pharynx. Swallowing is strongly impeded through the strong bracing at the back lower pharyngeal wall.

### REPRESENTATION OF THE INVENTION

The aim of the invention is to propose a device of the kind mentioned at the beginning, that is easier to produce, relatively cheap and that can serve for keeping the pharynx open, for stabilizing the velum from the pharynx space, for stabilizing the back of the tongue and keeping the back of the tongue from the pharynx back wall, for determining the place of the obstruction of snoring and sleep apnea patients, for determining the air flux in the pharynx, for training the throat and palate muscles in the case of sleeping, speaking and swallowing disorders. It should be independent of the dentition.

In order for it to be capable of being placed through the mouth in the pharynx without triggering an excessive gagging reflex, the device must be controllable from outside.

These aims are achieved according to the invention through the characteristics of the independent claims.

In particular, said aims are achieved with a device for treating snoring sounds, interruptions in breathing and obstructive sleep apnea (OSA) and for diagnosing and treating obstructions and vibrations in the pharyngeal region, with the device being used intra-orally, and with the device being comprised of at least one oral transverse and of means placed at the rear end of the oral transverse for stabilizing the velum; the means can be individually adapted to the patient's anatomy and during intra-oral use of the device, the velum is held at a distance from the pharyngeal wall and the connective tissue in the upper pharynx behind the velum is stabilized so that obstructions and vibrations are prevented.

The aims are also achieved with a method for adapting a device according to an inventive device to the velum of a patient, where the means for stabilizing the velum can be adapted in shape and height to the anatomy of the patient's velum.

The aims are also achieved with a computer program product that stores a software code capable of running on a computer to evaluate the generated images of the velum and of the pharynx of a patient and calculate and then display on the computer the optimum shape of the means for stabilizing the velum and/or an existing tongue relapse.

The aims are also achieved with a method for recording measured values with a device according to one of the preceding claims, where measurements of temperature, pressure, electrogram, head position, breath flux and/or acoustics are performed through sensors or probes that are placed on the device, where said sensors or probes are connected to external appliances and the evaluation of the recorded measurement data is carried out by the external appliances.

Advantageous embodiments of the present invention are indicated in the dependent claims.

The means for stabilizing the velum can, according to the invention, consist of a single or double pharynx arch, a fine meshing, a plate and/or a form that can be inflated or filled with a medium.

The oral traverses can be straight or round shaped or have a U shape and are fastened with reference shanks to the corners of the mouth or brought together directly in the mouth next to the rows of teeth, or fastened to braces made for this application.

The means for stabilizing the velum can be adjusted to the anatomy of the patient and can have different shapes and heights so that they can be formed harmoniously close to the patient's velum. Conceivable are a ∩ shape or an Ω shape with a variable radius, an M shape, an inverse V shape, a heart shape, an Ω shape with an additional upper M or other shapes that can serve the same purpose. They can be bent backwards in order to additionally support the throat wall. Advantageously, a double pharynx arch can be used, where each pharynx arch can be or is controlled separately from the end of the oral traverses or from the reference shanks.

With a tongue relapse between the oral traverses and the means for stabilizing the velum, an additional tongue immobilization can be achieved retro-lingually. This is particularly advantageous for persons whose snoring and/or apnea problems are caused through the tongue relapsing.

Additional means can be present on the device for stabilizing the tissues higher up next to the nose septum.

The invention can further be executed not only in one piece but also of several parts. For example inserted or affixed gnashing protection on the tooth contact surface, multipart constructions in which the velum-stabilizing means, the oral traverses and the reference shanks are made fully or partly of another material than the rest of the construction, are possible.

The device consists in one shape of a plastic tube that contains inside a flexible wire. It is also possible in this case to provide for use of fully or partly spiral-shaped twisted spring wire in order to achieve fine nuances in hardness and flexibility. The construction of an air gap sheathed in plastic is also conceivable.

In a further embodiment of the inventive braces, means such as sensors or probes for the measuring of movement, temperature, pressure, electrogram, head position and acoustics can be integrated. Alternatively or additionally, the sheathing can also be perforated so that the measuring instruments for measuring the breathing flux, the pressure conditions, the muscle activity and the position of the head in the pharynx and for measuring noises. The sensors can in principle be fastened to the means for stabilizing the velum, to the front end of the oral traverses or to the reference shanks. For this purpose, electric connections to the reference shanks (or at the front end of the oral traverses) are provided that are connected with the sensors. External appliances perform the evaluation of the measured data in known manner.

A further embodiment for therapeutic purposes concerns an installation for training the throat and palate muscles in the case of sleeping, speaking and swallowing disorders. A balloon is affixed to the pharynx arch or to the other said means for stabilizing the velum and can be rapidly filled with air or with liquid and thus it is possible to train palate muscles with swallowing movements.

In the case of the invention described herein, the problem of snoring and sleep apnea is addressed directly at the point of origin in the pharynx and at the velum. The construction is easy to make, is cost-effective and also hardly causes the patients to vomit. It is easy to insert into the mouth as long as the braces have been adapted by a specialist to the patient's anatomy. Afterwards, the patient can put on the braces without problems before going to bed and easily take them out again after getting up. These braces have yielded very good results in tests with the easy means described. Cleaning is also very easy, as it is of plastic that is easy to wipe and dry. Measurements for example in a sleep lab can advantageously be taken directly at the point of origin, by connecting the external appliances to the braces.

### BRIEF DESCRIPTION OF THE DRAWINGS

The invention will be described in more detail on the basis of the attached figures, which show:

FIG. 1 a first embodiment of inventive braces resp. device;

FIG. 2 a top view of the arrangement of inventive braces in the mouth, where the oral traverses are directed to the side of the teeth;

FIG. 3 a lateral view of the arrangement of inventive braces in the mouth with a tongue depressor;

FIG. 4 the movement sequence when inserting the braces in the mouth;

FIG. 5.1-5.3 different embodiments to adapt the pharynx arch to the patient's anatomy;

FIG. 6 an embodiment of inventive braces with a double pharynx arch;

FIG. 7 an embodiment of inventive braces with distension measuring resistances in the pharynx arch and electric connections;

FIG. 8 an embodiment of inventive braces with a perforation at least in the pharynx arch to affix different sensors or probes either directly in the perforation at the front end of the oral traverses or at the reference shanks;

FIG. 9 an embodiment of inventive braces with a bellows and an inflatable balloon;

FIG. 10 a diagrammatic representation of the application of the embodiment of FIG. 9 in the mouth and throat area of a patient;

FIG. 11 an embodiment of inventive braces with affixed gnashing protection;

FIG. 12 an embodiment of inventive braces fastened to a tooth plate, and

FIG. 13 an embodiment of an inventive device with a fine meshing or a plate as a means for stabilizing the velum.

### WAYS OF EXECUTING THE INVENTION

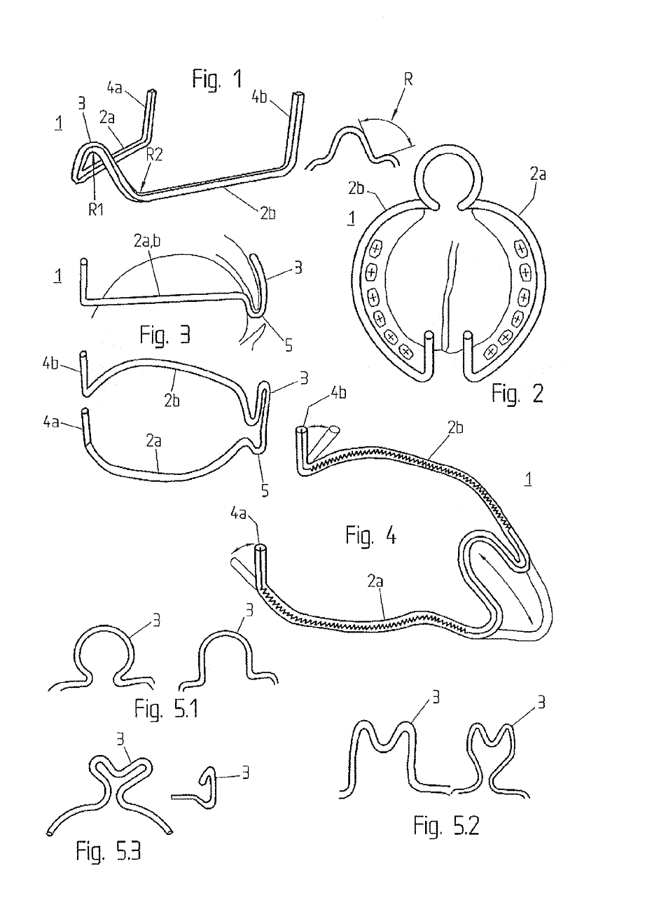

A first embodiment of inventive braces resp. device 1 can be seen in FIG. 1. It consists of two lateral oral traverses 2a, 2b and of a pharynx arch 3 placed at the rear end of the oral transverses 2a, 2b. During intra-oral use, the pharynx arch 3 is placed behind the velum and the velum is supported from the throat (pharynx) and held at a distance from the pharyngeal wall, so that the airways are kept free by the inventive device 1 during the patient's sleep. Simultaneously, the connective tissue in the upper pharynx behind the velum is stabilized in order to prevent obstructions.

In all embodiments of the inventive device 1, besides one or a double pharynx arch 3, a fine a fine meshing 14 (FIG. 13), a plate (FIG. 15) and/or an inflatable or fillable form can be used as means for stabilizing the velum, combinations thereof being also possible. An embodiment with a fine mesh 13 or a plate 14 can be seen in FIG. 13. Other means that fulfill the same purpose of stabilizing the velum and the connective tissue located above the velum can however also be used within the frame of the present invention.

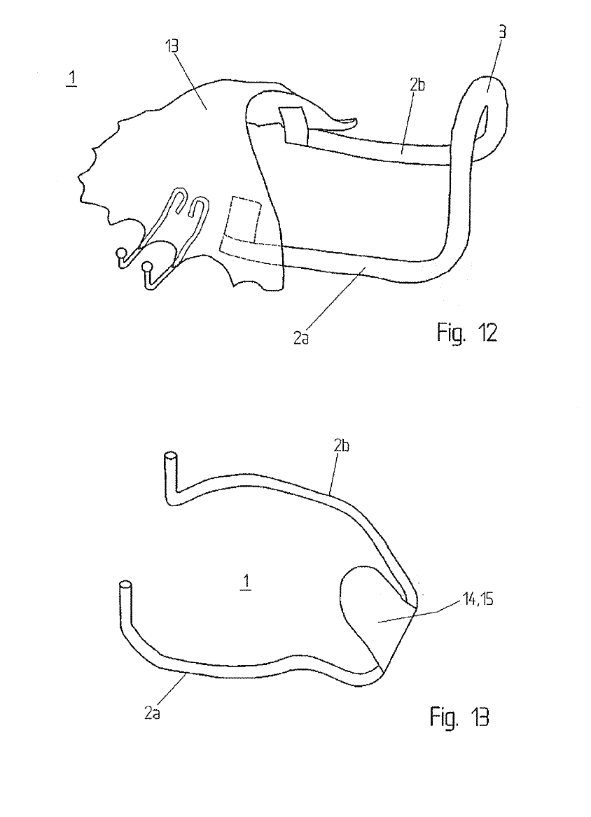

With the inventive device 1, there is no snoring and obstructive sleep apnea can be massively reduced or fully prevented. The device 1 is fastened outside the mouth with two reference shanks 4a, 4b at the front end of the oral traverses 2a, 2b. The fastening can however also occur through other suitable means, and it is thus conceivable to have an elastic band fastened at both oral traverses 2a, 2b that is placed around the head. In another embodiment, the oral traverses 2a, 2b are brought together distally over the upper teeth rows and fastened inside the mouth. The oral traverses 2a, 2b can also be fastened to a tooth plate 13 inside the mouth, as can be seen in the embodiment of FIG. 12.

The oral traverses 2a, 2b that pass through the mouth lead in a straight way from the corners of the mouth over the lateral teeth rows to the arci palatopharyngae, where the oral traverses 2a, 2b open up into an arc with a radius R₂ of 120° in the inverted V-shaped or ∩ shaped pharynx arch 3. The pharynx arch 3 has a variable radius R₁.

The device 1 of FIG. 1 is in a simple embodiment made of a PVC-wire-construction and leads directly over the teeth and tongue. In its simples form, it consists of a plastic tube that contains inside it a flexible wire. As can be seen in FIG. 4, in a further embodiment the use of fully or partly spiral-shaped twisted spring wire as part of the forming construction, in order to achieve fine nuances in hardness and flexibility of the device 1 and make it easier to adapt the device 1 to the patient's anatomy. The construction of an air gap sheathed in plastic is also conceivable.

The device 1 can also consist of other well-tolerated and flexible as well as sufficiently stable materials. The forming construction can also be fully molded in plastics. The sheathing of the forming construction can also be completely or partly filled with a material capable of flowing. A further embodiment comprises that the device or parts thereof are covered with a well-tolerated material capable of flowing in order to prevent pressure points in the throat and improve tolerance.

FIG. 2 shows the view from above of the arrangement of inventive braces in the mouth. In this embodiment, the oral traverses 2a, 2b can pass next to the rows of teeth. They are therefore made in the U-shape required for this or are generally made round. In another embodiment, the oral traverses 2a, 2b are brought together distally over the upper teeth rows and fastened inside the mouth. In this version, the intra-oral fastening can take on the task of the mentioned reference shanks 4a, 4b.

FIG. 3 shows a lateral view of an embodiment of inventive braces 1 in the mouth. For persons whose snoring and/or apnea problems are caused or exacerbated by the tongue relapsing, a tongue depressor 5 can be provided in this embodiment under the arci palatopharyngae.

FIG. 4 shows the movement sequence for intra-orally inserting the braces 1. In order for the construction to be placed through under the uvula into the pharynx without touching the spots particularly sensitive for the gagging reflex, the pharynx arch 3 can be controlled resp. flattened by means of the reference shanks 4a, 4b.

The pharynx arch 3 is, in its simplest form, similar to an inverted V (see FIG. 1). In order to meet the requirements of individual anatomy and problems of particular individuals, embodiments as ∩ shape or as Ω shape (FIG. 5.1) with a variable radius R₁ (FIG. 1), as M shape (FIG. 5.2) or as inverse V shape, or as heart (♡) shape, an Ω shape with an additional upper M (FIG. 5.2) or other shapes are conceivable. For example, the pharynx arch 3 can also be bent backwards in order for it to also support the throat back wall, which gives the construction additional support (FIG. 5.3). Additional means can furthermore be provided for stabilizing the tissues higher up next to the nose septum.

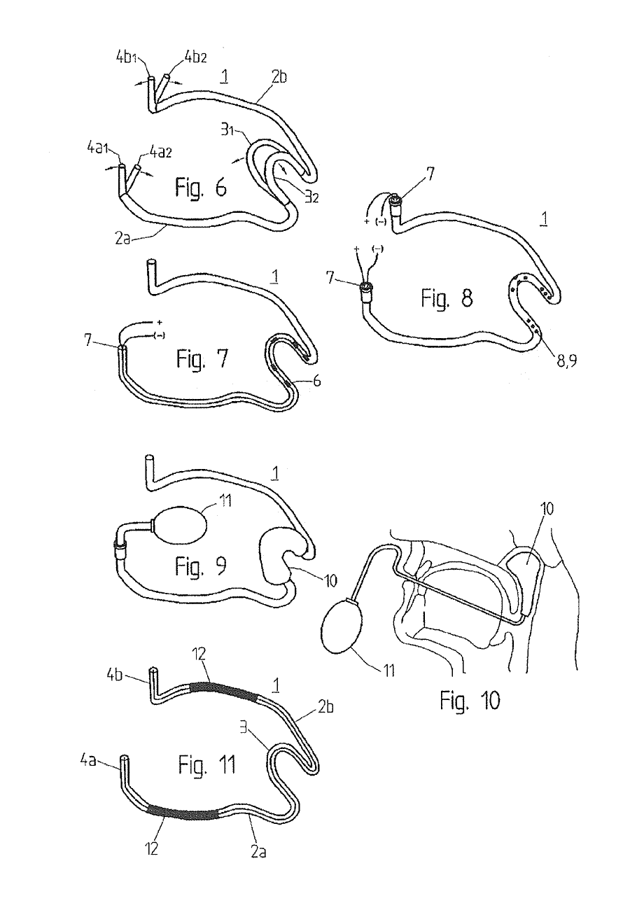

FIG. 6 shows an embodiment of inventive braces 1 with double pharynx arches 3₁, 3₂, one arch 3₁ stabilizing the velum and the other arch 32 resting against the throat's back wall. Both arches 3₁, 3₂ can also be controlled independently from one another through a double construction with double reference shanks 4a₁, 4b_{1,} 4a₂, 4b₂.

As can be seen in FIG. 7, means for specific applications such as for the measuring of movement, temperature, pressure, electrogram (e.g. electromyogram), head position and acoustics can be integrated in the pharynx arch 3 in further embodiments of the inventive braces 1. Alternatively or additionally, as represented in FIG. 8, the sheathing in the pharynx arch 3 or on the other said means for stabilizing the velum can also be provided with perforations 8 so that the measuring instruments (sensors 6, 9, measuring probes, etc.) for measuring the breathing flux, the pressure conditions in the pharynx and noises can be connected. The sensors 9 can also be fastened to the front end of the oral traverses 2a, 2b and/or to the reference shanks 4a, 4b. For the purpose of connecting the appliances, connecting cables are lead through the oral traverses 2a, 2b that open out at the end of the oral traverses 2a, 2b or at the reference shanks 4a, 4b in electric connections 7. To these connections 7, external appliances are connected (not represented in the figures) that perform the evaluation of the measured data in known manner. Of course, said sensors 6, 9 or probes can also be connected directly to the pharynx arch 3 to external appliances.

A further embodiment for therapeutic purposes concerns an installation for training the throat and palate muscles in the case of sleeping, speaking and swallowing disorders. According to FIGS. 9 and 10, a balloon 10 is affixed to the pharynx arch 3 or to the other said means for stabilizing the velum and can be rapidly filled with air or with liquid and thus it is possible to train palate muscles with swallowing movements. In order to pump up the balloon 10 from outside, a small hand pump resp. a hand operated bellows 11 is provided that is fastened to a connection on one of the reference shanks 4a, 4b. The air or liquid can then reach the balloon 10 through the oral traverses 2a, 2b and blow it up.

The device 1 can further be executed not only in one piece but also of several parts. For example inserted or affixed gnashing protection 12 on the tooth contact surface (FIG. 11) or multipart constructions in which the pharynx arch 3 or the other velum stabilizing means, the oral traverses 2a, 2b and the reference shanks 4a, 4b are made fully or partly of another material than the rest of the construction.

To adapt the means 3 for stabilizing the velum, image-producing means such as for example a miniaturized camera can be introduced intra-orally and into the pharynx of a patient. In this manner, images of the velum of the future use of the device 1 are recorded and then transmitted to a computer. With the aid of a software running on a computer, the optimum shape of the means 3 for stabilizing the velum for an individual person can be computed from the recorded images and represented. The same is also valid for a possibly existing tongue relapse 5. To make an inventive device 1, hand-operated or automatic devices such as a bending caliper can be used.

Accordingly, the present invention also concerns a computer program product that stores a software code capable of running on a computer to evaluate the generated images of the velum and of the pharynx of a patient and calculate and then display the optimum shape of the means 3 for stabilizing the velum and/or an existing tongue relapse 5.

In the case of the invention described herein, the problem of snoring and sleep apnea is addressed directly at the point of origin in the pharynx and at the velum. The construction is easy to make, is cost-effective and, contrary to received opinion, also hardly causes the patients to vomit. It is easy to insert into the mouth as long as the braces have been adapted by a specialist to the patient's anatomy. Afterwards, the patient can put on the braces 1 without problems before going to bed and easily take them out again after getting up. These braces have yielded very good results with the easy means described. Cleaning is also very easy, as it is of plastic that is easy to wipe and dry. Measurements for example in a sleep lab can advantageously be taken directly at the point of origin, by connecting the external devices to the braces.

### LIST OF THE REFERENCES

1 Device, braces
2a, 2b Oral traverses
3, 3₁, 3₂ Pharynx arches
4a, 4b Reference shanks
4a₁, 4b₁, 4a₂, Reference shanks
4b₂.
5 Tongue depressor
6 Measuring sensor
7 Connections
8 Perforation
9 Measuring sensor
10 Balloon
11 Hand operated bellows
12 Gnashing protection
13 Tooth plate
14 Fine meshing
15 Plate
R₁, R₂ Radius

1. Device for treating snoring sounds, interruptions in breathing and obstructive sleep apnea (OSA) and for diagnosing and treating obstructions and vibrations in the pharyngeal region, with the device being used intra-orally and where the device is comprised of at least one oral transverse and of means which are placed at the rear end of the oral transverse for stabilizing the velum, with the means being individually adaptable to the patient's anatomy and where during intra-oral use of the device the velum is held at a distance from the pharyngeal wall and the connective tissue in the upper pharynx behind the velum is stabilized in order to prevent obstructions.

2. The device of claim 1, characterized by one or a double pharynx arch, a fine mesh, a plate and/or an inflatable or fillable form as means for stabilizing the velum.

3. The device of claim 1, characterized in that the device consists of two lateral oral traverses, wherein the means for stabilizing the velum are provided at the rear end of the oral traverses.

4. The device of claim 1, characterized in that at the front end of the oral traverses reference shanks for fastening the device outside the mouth are provided at the corners of the mouth.

5. The device of claim 1, characterized in that means for fastening the oral traverses are provided inside the mouth.

6. The device of claim 1, characterized in that the oral traverses are shaped straight or round or have a U-shape.

7. The device of claim 1, characterized in that the device for immobilizing the tongue retro-lingually has a tongue depressor between the oral traverses and the means for stabilizing the velum.

8. The device of claim 1, characterized in that the means for stabilizing the velum can be controlled by manipulating the front end of the oral traverses or the reference shanks and can thus be placed through under the velum and the uvula from the pharynx.

9. The device of claim 1, characterized in that the oral traverses have gnashing protection.

10. The device of claim 1, characterized in that additional means are present on the device for stabilizing the tissues higher up than the velum next to the nose septum.

11. The device of claim 1, characterized in that the device has sensors or probes for recording measurements of movement, temperature, pressure, breath flux, electrogram, head position and/or acoustics.

12. The device of claim 1, characterized in that the device has a sheathing and at least the sheathing has perforations and sensors or probes for recording temperature, pressure, breath flux, electrogram, head position and/or noises are provided there or are affixed at the front end of the oral traverses or on the reference shanks.

13. The device of claim 11, characterized in that the sensors or probes can be connected to external appliances and the connections between said sensors or probes and the connections pass inside the device.

14. The device of claim 1, characterized in that the device consists if well-tolerated and flexible materials.

15. The device of claim 1, characterized in that the device is made in one piece or of several parts.

16. The device of claim 1, characterized in that the device is made of an air gap sheathed in plastic, a material capable of flowing, a wire or a fully or partly spiral-shaped twisted spring wire and/or is made totally of plastic.

17. The device of claim 1, characterized in that the device is sheathed at least partly with a well-tolerated material capable of flowing.

18. A method for adapting the device of claim 1 to the velum of a patient, where the means for stabilizing the velum can be adapted in shape and height to the anatomy of the patient's velum.

19. The method of claim 18, characterized in that a tongue depressor is provided between the oral traverses and the means for stabilizing the velum for immobilizing the tongue retro-lingually and the tongue depressor is also adapted to the patient's anatomy.

20. The method of claim 18, characterized in that image-producing means are inserted intra-orally and in the pharynx and in that with these generated images of the velum with the aid of a software the optimum shape of the means for stabilizing the velum and/or the tongue depressor can be computed and represented.

21. The method of claim 18, characterized in that the means for stabilizing the velum can be controlled by manipulating the front end of the oral traverses or the reference shanks and can thus be placed through under the velum and the uvula in the pharynx.

22. The method of claim 21, characterized in that a double pharynx arch is provided and each pharynx arch is controlled separately from the end of the oral traverses or from the reference shanks.

23. The method according to claim 18, characterized in that for making a device and for adapting the means for stabilizing the velum, hand-operated or automatic devices are used.

24. Computer program product that stores a software code capable of running on a computer to execute a method according to claim 20 and evaluate the generated images of the velum and of the pharynx of a patient and calculate and then display the optimum shape of the means for stabilizing the velum.
